(19) United States
(12) Patent Application Publication
(10) Pub. No.: US 2014/0135722 A1
(43) Pub. Date: May 15, 2014

(54) DISPOSABLE ABSORBENT PRODUCT WITH DETECTABLE INODOROUS COMPONENT, AND SYSTEMS INCLUDING SAME

(71) Applicant: SCA HYGIENE PRODUCTS AB, Goteborg (SE)

(72) Inventors: Eugene Patrick DOUGHERTY, Jr., Newark, DE (US); Frank Stephen GLAUG, Chester Springs, PA (US); Raymond Martin CARTER, Philadelphia, PA (US)

(73) Assignee: SCA HYGIENE PRODUCTS AB, Goteborg (SE)

(21) Appl. No.: 13/674,980

(22) Filed: Nov. 13, 2012

(51) Int. Cl. A61F 13/42

(52) U.S. Cl. CPC A61F 13/42
USPC 604/361

(57) ABSTRACT

A disposable absorbent product includes a topsheet, a backsheet overlaying the topsheet, and an absorbent core disposed between the topsheet and the backsheet for absorbing fluid secreted by a wearer of the product. The product also includes a wetness indicating material in the topsheet, the backsheet, or the absorbent core, with the wetness indicating material having a water-soluble element and an inodorous component trapped by the water-soluble element. The water-soluble element is configured to degrade in the presence of fluid secreted by the wearer of the product, thereby releasing the inodorous component into the surrounding air.

### TECHNICAL FIELD

The present invention is generally related to absorbent products and, more particularly, to disposable absorbent products having wetness indicating features.

### BACKGROUND

Disposable absorbent products, such as baby diapers, training pants, adult briefs, animal diapers, disposable swimming underwear, and incontinent pads, are typically designed to retain fluid bodily waste such as watery feces, urine, blood, or menses secreted by the person or animal wearing the disposable absorbent product. It is often desirable to detect when the product has been insulted with a fluid of this type, so as to avoid prolonged exposure of the wearer's skin to those fluids, or even to avoid leakage if the product has reached its maximum holding capacity.

To that end, diapers and similar products have been provided with inks or other components that change color upon their exposure to urine. But products of this type rely on the visual acuity of the caretaker, which may not be suitable to detect the color change. And detection in the dark is often nearly impossible, due to the absence of light. In that regard, for example, a mother is not able to use that type of wetness-indicating feature to detect whether her child's diaper has been insulted with urine while the child sleeps at night.

Other products have been provided with pouches containing an odorous agent that is released in the presence of urine. The odorous agent in some cases emits a foul odor, which is intended to encourage the wearer, such as a child of toilet-training age, to control the secretion of urine so as to avoid a similar occurrence in the future. In other cases, the odorous agent emits a pleasant odor, which acts as a recognizable signal to the caretaker that urine is present in the product. A problem with products of this type, however, is that the emitted odor, whether pleasant or foul, eventually becomes annoying to the wearer and/or caretaker. This annoyance makes the product less appealing and even undesirable.

Yet other products have been provided with a built-in electronic circuit that is closed in the presence of bodily fluids, such as urine. When the circuit is closed, an alarm may be triggered alerting the wearer or a caretaker that the product has been insulted with fluid. But these products are complex and often difficult to manufacture in high-speed manufacturing lines.

It is therefore desirable to provide disposable absorbent products that provide a way for indicating when a product-insulting event has taken place, while addressing the shortcomings described above.

### SUMMARY

In one embodiment, a disposable absorbent product is provided. The product includes a topsheet, a backsheet overlaying the topsheet, and an absorbent core disposed between the topsheet and the backsheet for absorbing fluid secreted by a wearer of the product. The product also includes a wetness indicating material in the topsheet, the backsheet, or the absorbent core, with the wetness indicating material having a water-soluble element and an inodorous component trapped by the water-soluble element. The water-soluble element is configured to degrade in the presence of fluid secreted by the wearer of the product, thereby releasing the inodorous component into the surrounding air.

The wetness indicating material may be embedded in the absorbent core. Additionally or alternatively, the water-soluble element may include a layer of a water-soluble substrate, with the inodorous component being in or located adjacent the substrate. In specific embodiments, the water-soluble element includes first and second water-soluble substrates, and the inodorous component is located between the first and second substrates. The inodorous component may be encapsulated within shells of the water-soluble element. In specific embodiments, the inodorous component is a liquid at room temperature. Alternatively, the inodorous component may be a gas at room temperature. For example, the inodorous component may be an inert gas. Alternatively, the inodorous component may include at least one gas selected from the group consisting of nitrogen, hydrogen, oxygen, methane, a nitrogen oxide, carbon dioxide, ethane, ethylene, propane, butane, isobutene, and acetylene. Yet in other specific embodiments, the water-soluble element includes a material selected from the group consisting of zeolites and hemicarcerands.

In another embodiment, a system is provided for managing body waste. The system includes at least one disposable absorbent product, and a sensing device in proximity with the disposable absorbent product. The disposable absorbent product includes a topsheet, a backsheet overlaying the topsheet, and an absorbent core disposed between the topsheet and the backsheet for absorbing fluid secreted by a wearer of the at least one disposable absorbent product. The disposable absorbent product also has a wetness indicating material in the topsheet, the backsheet, or the absorbent core, with the wetness indicating material including a water-soluble element and an inodorous component trapped by the water-soluble element. The water-soluble element is configured to degrade in the presence of fluid secreted by the wearer, to thereby release the inodorous component into the surrounding air. The sensing device is configured to detect the presence of the inodorous component in the surrounding air upon degradation of the water-soluble element, and to generate a signal in response to the detection.

In specific embodiments, the system also includes an alarm device that is operatively coupled to the sensing device, and which is actuatable in response to the signal generated by the sensing device. The sensing device and the alarm device may form part of an integrated apparatus. Alternatively, the alarm device and the sensing device may be separate apparatus. For example, the alarm device may be located remotely from the sensing device. In specific embodiments, the alarm device is configured to emit, when actuated, at least one of a visual display, an auditory signal, or a vibratory signal. The system may additionally have a controller that is operatively coupled to the sensing device and to the alarm device. In that embodiment, the controller is configured to actuate the alarm device only when at least a predetermined threshold level of inodorous component has been detected by the sensing device. The inodorous component may be a gas at room temperature, with the gas in specific embodiments being encapsulated within shells of the water-soluble element.

In yet another embodiment, a disposable absorbent product is provided. The product includes a topsheet, a backsheet overlaying the topsheet, and an absorbent core disposed between the topsheet and the backsheet for absorbing fluid secreted by a wearer of the product. The product also includes a wetness indicating material in the topsheet, the backsheet, or the absorbent core, with the wetness indicating material having a water-soluble element and an inodorous gas encapsulated by the water-soluble element. The water-soluble element is configured to dissolve in the presence of fluid secreted by the wearer of the product, thereby releasing the inodorous gas into the surrounding air. In specific embodiments, the gas is an inert gas. The water-soluble element may include a material selected from the group consisting of zeolites and hemicarcerands.

In another embodiment, a disposable absorbent product is provided that includes an absorbent core configured to absorb fluid secreted by a wearer of the product. The product also includes a wetness indicating material, in or adjacent the absorbent core, that includes a water-soluble element and an inodorous component trapped by the water-soluble element. The water-soluble element is configured to degrade in the presence of fluid secreted by the wearer of the product, thereby releasing the inodorous component into the surrounding air.

### BRIEF DESCRIPTION OF THE DRAWINGS

The objectives and features of the invention will become more readily apparent from the following detailed description taken in conjunction with the accompanying drawings in which:

FIG. 1 is a perspective, partially schematic view of a system for managing body waste, in accordance with one embodiment of the invention.

FIG. 2 is a perspective, partially schematic view of an exemplary disposable absorbent product forming part of the system of FIG. 1.

FIG. 3 is a partially schematic top view of the product of FIG. 2.

FIG. 4 is a schematic representation of a wetness indicating material, in accordance with one embodiment of the invention.

FIG. 5 is a schematic representation of a wetness indicating material, in accordance with another embodiment of the invention.

FIG. 6 is a schematic representation of a wetness indicating material, in accordance with yet another embodiment of the invention.

FIG. 7 is a schematic representation of an exemplary process for managing body waste.

FIG. 8 is a schematic representation of an apparatus forming part of a system for managing body waste, in accordance with another embodiment of the invention.

FIG. 9 is a view similar to FIG. 7, schematically illustrating another exemplary process for managing body waste.

FIG. 10 is a schematic representation of a portion of a system for managing body waste, in accordance with another embodiment of the invention.

FIG. 11 is a view similar to FIGS. 7, 9, and 10, schematically illustrating yet another exemplary process for managing body waste.

FIG. 12 is a perspective, partially schematic view of a disposable absorbent product in accordance with another embodiment of the invention.

### DETAILED DESCRIPTION

To the extent that any meaning or definition of a term in this written document conflicts with any meaning or definition of that term in a document incorporated by reference, the meaning or definition assigned to that term in this written document shall govern. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

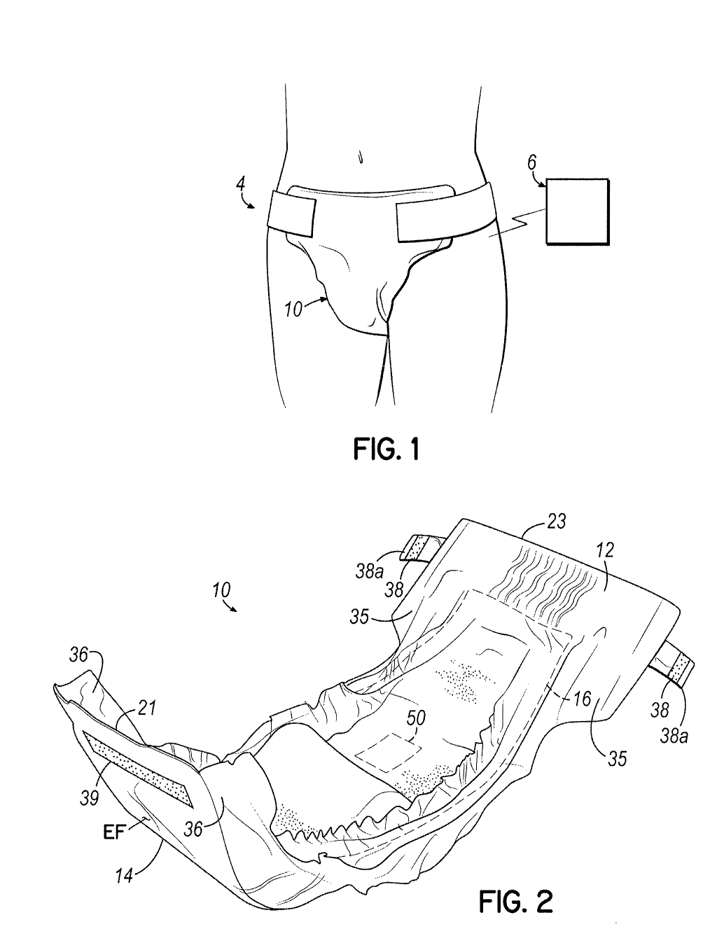

With reference to the figures, and more particularly to FIG. 1, a system 4 is illustrated for managing body waste, such as watery feces, urine, blood, or menses. System 4 includes at least one disposable absorbent product, such as a diaper 10 or another type of disposable absorbent product (e.g., sanitary napkin, disposable swimwear, light or heavy incontinence pad, tampon, training pant, adult brief) and a schematically-illustrated sensing device 6. As explained more fully below, diaper 10 includes features that cause the release of an inodorous component upon being exposed to fluids secreted by the wearer of the diaper 10, while sensing device 6 is configured to detect the presence of the inodorous component in the surrounding air and to generate a signal in response to the detection.

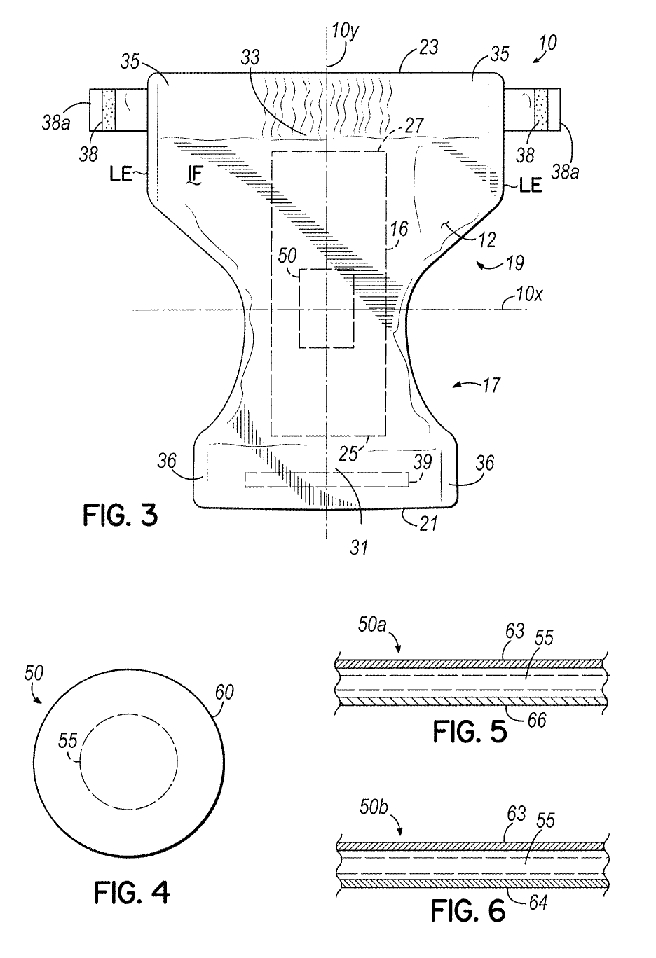

Referring now to FIGS. 2 and 3, those figures illustrate additional details about exemplary diaper 10. For ease of understanding, some of the features of diaper 10 shown in FIG. 2 are obviated from FIG. 3. Diaper 10 could be a baby diaper or an adult diaper (“brief”), for example. Likewise, diaper 10 could be a training pant to be used for toilet-training a child, with the present disclosure being in that regard similarly applicable to open and closed (i.e., pant-like) diapers. The exemplary diaper 10 of FIGS. 2 and 3 extends in the longitudinal direction along a longitudinal axis 10y, and extends laterally along a transverse axis 10x orthogonal to the longitudinal axis 10y, as illustrated in FIG. 3. Diaper 10 has a topsheet 12 and a backsheet 14 disposed opposite the topsheet 12, such that the topsheet 12 and backsheet 14 are in an overlaying relationship with one another. An absorbent core 16 is disposed between the topsheet 12 and backsheet 14, as more fully explained below. In use, the topsheet 12 at least partially defines an interior face IF of the diaper 10 that faces the body of the wearer, in use. The backsheet 14, in contrast, faces away from the body of the wearer, in use, and accordingly at least partially defines an exterior face EF of the diaper 10.

The topsheet 12 is at least partially made of a permeable, hydrophilic material such as a hydrophilic nonwoven, and may be in the form of a single, continuous layer spanning across the length and/or width of the diaper 10, or may alternatively be in the form of two or more layers of the same material or of materials different from one another that jointly, rather than individually, span the length and/or width of the diaper 10. In that regard, the term “topsheet” as used herein can refer to a single layer that lies over the absorbent core 16 as well as partially or entirely over regions laterally outboard of the core 16, or can alternatively refer to a plurality of layers of material joined to one another so as to span the entire width of diaper 10.

The backsheet 14 is at least partially made of an impermeable, hydrophobic material, such as a hydrophobic nonwoven or a laminate made of one or more layers of nonwoven material and one or more layers of polypropylene or polyethylene film. Backsheet 14 may be in the form of a single, continuous layer spanning across the length and/or width of the diaper 10, or may alternatively be in the form of two or more layers of the same material or of materials different from one another that jointly, rather than individually, span the length and/or width of the diaper 10.

The diaper 10 also includes, as discussed above, an absorbent core 16, disposed between the topsheet 12 and the backsheet 14. The core 16 is configured to absorb and retain bodily fluids, such as watery feces, urine, blood, and/or menses, secreted by the wearer. The core 16 may be made up of fluff pulp or a combination of fluff pulp or some other natural or synthetic fluid management material, and a fluid storage material such as superabsorbent material (“SAP”) or some other natural or synthetic fluid storage material. While not shown, core 16 may also include an optional acquisition material layer or another type of layer (e.g., an airlaid material layer) adjacent the topsheet 12, which is primarily configured to distribute and/or direct fluids received through the topsheet 12 onto other portions of core 16 that are primarily configured to store fluids secreted by the wearer. The core 16 could be generally rectangular, or have an hourglass shape, or have any other regular or irregular, symmetrical or asymmetrical shape.

With continued reference to FIGS. 2 and 3, the transverse axis 10x conceptually divides the diaper 10 into front and back longitudinal hemispheres 17, 19. The diaper 10 thus extends longitudinally between a front longitudinal end 21 and a back longitudinal end 23. The core 16, in turn, extends longitudinally between a front edge 25 and a back edge 27. While the front and back edges 25, 27 are illustrated in the figures as generally rectilinear, it is understood that either or both may have any other regular or irregular shape, such as one including curves, straight line segments or any other shape. The front longitudinal end 21 and the front edge 25 fall generally within the front portion of the diaper 10 which, in use, generally faces the front part of the body of the wearer. In contrast, the back longitudinal end 23 and the back edge 27 fall generally within the back portion of the diaper 10, which generally faces the back part of the body of the wearer. In this regard, and as used herein, the terms “front portion” and “back portion,” when used to describe parts of the diaper 10, do not imply the existence of any specific structural boundaries within the diaper 10, but instead refer to the general areas that, in use, lie respectively against the front and back parts of the body of the wearer. In that regard, the diaper 10 is also considered to include a crotch portion located between the front and back portions thereof, which is a region of diaper 10 that generally lies against the crotch of the wearer, in use.

The core 16 of the illustrated embodiment is positioned, relative to other portions of the diaper 10, such that a front end portion 31 is defined between the front longitudinal end 21 and the front edge 25 of the core 16. The exemplary core 16 is also positioned so as to define a back end portion 33 between the back longitudinal end 23 and the back edge 27 of the core 16. Those of ordinary skill in the art will readily appreciate, however, that other relative positions of the core 16 are possible which may, for example, define only one end portion 31, 33 or define no such end portions at all. In other words, alternative configurations are contemplated in which the core 16 may instead extend to one or both of the longitudinal ends 21, 23.

With continued reference to FIGS. 2 and 3, diaper 10 includes a pair of side regions in the form, in this embodiment, of side panels 35, laterally outboard of the core 16 in the back portion of the diaper 10. The side panels 35 are thus made up of portions of the topsheet 12 and backsheet 14 that are joined together with adhesive elements, and/or mechanically (e.g., CPW or ultrasonic bonding), for example, so as to form an integral structure. In specific embodiments, the side panels 35 may include elastomeric portions (not shown) that allow the side panels 35 to laterally stretch with relative ease, to accommodate convenient application of diaper 10 and provide comfort to the wearer. The elastomeric portions may in some embodiments extend into areas directly beneath the core 16. The exemplary diaper 10 similarly has another pair of side regions 36, also laterally outboard of core 16, but located generally in the front portion of the diaper 10. The construction of the side regions 36 is generally similar to that of the side panels 35, which may for example optionally include elastomeric portions (not shown) similar to or different from the optional elastomeric portions in side panels 35.

Diaper 10 has a pair of fastening components such as mechanical fasteners or adhesive or cohesive tapes, generally assigned the numeral 38, located in the back portion of diaper 10. In the illustrated embodiment, the fastening components 38 are supported on respective tabs 38a extending generally from the lateral ends LE of the diaper 10. Other embodiments are also contemplated within the scope of the present disclosure, in which the fastening components 38 are supported directly on the side panels 35, rather than on tabs, and located generally adjacent the lateral ends LE of diaper 10.

The fastening components 38 are engageable with a cooperating fastening component or feature 39 in the front portion of the diaper 10 to secure the diaper 10 in place, on the body of the wearer. The cooperating fastening feature 39 may for example be in the form of an adhesive or cohesive landing zone or patch, or a mechanical fastener landing zone or patch (e.g., containing hooks or loops) coupled (e.g., mechanically and/or adhesively attached) to the backsheet 14 and which cooperates with fastening components 38 to secure the diaper 10 in place, on the body of the wearer. In the exemplary embodiment of FIGS. 2 and 3, the fastening components 38 include hook members and the cooperating fastening feature 39 includes a loop member. Entangled engagement of the hook and loop members with one another secures the front and back portions of diaper 10 to one another on the body of the wearer. Other embodiments are similarly contemplated in which the fastening components 38 are loop members that are engageable with a hook-type fastening feature 39. Yet other embodiments are contemplated in which fastening feature 39 includes two or more hook fasteners or loop fasteners, rather than a single structure (e.g., as in the exemplary single structure making up fastening feature 39 in FIGS. 2 and 3).

Fastening feature 39 may alternatively be defined by the exterior surface of the backsheet 14, so long as that surface is configured to directly entangle the fastening component 38 sufficiently so as to secure the diaper 10 in place i.e., on the body of the wearer. While the embodiment of FIGS. 2 and 3 has the fastening components 38 located in the back portion of diaper 10 and the cooperating fastening feature 39 located in the front portion, embodiments are contemplated in which the fastening components 38 are instead located in the front portion of diaper 10, while the cooperating fastening feature 39 is located in the back portion of diaper 10, so long as they are positioned so as to engage one another to secure the diaper 10 in place on the body of the wearer.

As noted above, diaper 10 includes features that indicate the presence of fluid secreted by the wearer of diaper 10. More specifically, diaper 10 includes a wetness indicating material 50 that is operative to release an inodorous, yet detectable component when diaper 10 has been insulted with fluids secreted by the wearer. For ease of understanding, the wetness indicating material 50 is schematically represented in FIGS. 2 and 3, being understood that wetness indicating material 50 is in no way limited to the specific dimensions, shape, or location thereof in those figures.

With continued reference to FIGS. 2 and 3, and further referring to FIGS. 4 and 5, additional details of wetness indicating material 50 are provided. Material 50 includes an inodorous component 55 that is trapped by a water-soluble element 60, with the entrapment being effective to prevent the release of the inodorous component 55 in the absence of urine or other wearer-secreted fluids in diaper 10. As used herein, the term “inodorous” is intended to describe components that are virtually undetectable by the human nose i.e., the human sense of smell. In that regard, “inodorous” components include components that are free of scents that are pleasant or unpleasant to humans, and further free of scents that are otherwise detectable by the human nose.

In specific embodiments, the inodorous component 55 is in gaseous form at room temperature i.e., in the range between about 20° C. and about 22° C. An exemplary gas making up the inodorous component 55 includes, without limitation, at least one gas selected from the group consisting of nitrogen, hydrogen, oxygen, methane, carbon dioxide, ethane, ethylene, propane, butane, isobutene, acetylene, nitrogen oxides (NₓO_{y}, where 0<x≦2 and 0<y≦5), and any combinations thereof. Other exemplary gases that are contemplated for inodorous component 55 are inert gases i.e., helium, argon, krypton, neon, radon and xenon. Yet in other embodiments, the inodorous component 55 is made up of or otherwise includes an oil or some other element that is in a liquid state at room temperature, but which nevertheless is relatively volatile so as to emit odorless vapors at room temperature. Exemplary materials of this type include but are not limited to ethanol, isopropanol, propanol, butanol, isobutanol, pentanol (and variants thereof, such as isopentanol), hexanol, acetic acid, low molecular weight silicones such as dimethicones, glycerol, ethylene glycol, triethanolamine, boric acid, sorbitol, benzoic acid, lactic acid, propylene glycol, soybean oil, safflower oil, and mineral oil.

Other exemplary materials suitable for inodorous component 55 include materials that are typically found in a solid state at room temperature. These materials include, without limitation, citric acid, beta carotene, cyclopentasiloxane, disteardimonium hectorite, cocamidopropyl betaine, diazolindinyl urea, Poloxamer 407, d-alpha tocopherol, polyethylene glycol, behenyl alcohol, disodium ethylene diamine tetraacetic acid (disodium EDTA), trisodium ethylene diamine tetraacetic acid (trisodium EDTA), tetrasodium ethylene diamine tetraacetic acid (trisodium EDTA), xanthan gum, pseudoephedrine, dipropylene glycol, isopropyl myristate, hydrolyzed milk protein, methylchloroisothiazolinone, methylisothiazolinone, cetyl alcoholsodium lauryl sulfate, Laureth-23, glycerol monostearate, stearic acid/sodium stearate, sodium dodecyl benzene sulfonate, sodium diocytyl sulfo succinate, erucamide, stearamide, sodium saccharin, diazomethane, sodium carbonate, and sodium bicarbonate. These last two exemplary materials are known to release carbon dioxide.

The water-soluble element 60 trapping the inodorous component 55 is suitably chosen so as to effectively trap the inodorous component 55, unless exposed to urine, menses, or other fluids secreted by the person wearing diaper 10. In that regard, the water-soluble element 60 may be a hemicarcerand, for example. Another example of a material suitable for water-soluble element 60 is a zeolite, such as a zeolite known as Ludox® LS-30, commercially available from Du Pont, of Wilmington, Del., United States of America. Yet other examples include carbon-based fullerenes, clathrates, cyclodextrins, cryptophanes, polysaccharides, carcerans, and calixarenes.

FIG. 4 schematically illustrates an exemplary wetness indicating material 50 that includes an inodorous component 55 encapsulated, and more specifically microencapsulated, by a shell or cage of the water-soluble element 60. In that regard, exposure of the cage to urine, menses, or other fluids secreted by the wearer is effective to at least partially dissolve or otherwise degrade the cage. This dissolution or otherwise degradation of the cage releases, into the surrounding air, the inodorous component 55 that is at the core of the microcapsules defining the wetness indicating material 50. As used herein, the term “dissolve” and derivatives thereof refer to the partial or complete change in phase, from solid to liquid, of the water-soluble element 60, so as to release the bonds or other constraints or restraints upon the inodorous component 55. As used herein also, the term “degrade” and derivatives thereof are intended to be broader than and encompassing “dissolve” or derivatives thereof, in the sense that dissolution of the water-soluble component 60 is intended to mean a type of degradation of that component. In that regard, it is also understood that degradation of water-soluble component 60 may involve physical or chemical processes other than dissolution, so long as that degradation results in the release of the bonds or other constraints or restraints upon the inodorous component 55.

In specific embodiments, the microcapsules defining the exemplary wetness indicating material 50 are incorporated into the core 16 of diaper 10 in a predetermined amount, pattern, and/or location. Alternatively, the microcapsules may be incorporated in an arbitrary amount, without a specified pattern, and/or at a random location in core 16. Further, the microcapsules may be, in specific embodiments, intermixed with SAP or other granular components that are incorporated into the diaper 10 in ways known to those of ordinary skill in the art.

FIG. 5 illustrates another embodiment in which a wetness indicating material 50a is made up of a layer of a water-soluble substrate 63 trapping the inodorous component 55 against an adjacent layer of a material, generally assigned the numeral 66, forming part of the diaper 10. For example, it is contemplated that the substrate 63 may be a water-soluble film or some other water-soluble material that is coated onto the layer 66, thereby trapping the inodorous component 55 there between. The layer 66 may, for example, be the backsheet 12, topsheet 14, or core 16. More specifically, the layer 66 may for example refer to the optional acquisition material layer of core 16, discussed above with respect to the overall construction of diaper 10. In specific embodiments, the substrate 63 is a water-soluble film or some other water-soluble material that is impregnated with particles or droplets of the inodorous component 55, and which is then joined to the layer 66. Generally, the location of wetness indicating material 50a is suitably chosen to be in or generally adjacent the core 16, so as to expose that material 50a to fluid secreted by the wearer. Exposure to urine, menses or other fluids secreted by the wearer is effective to dissolve or otherwise degrade the substrate 63, thereby permitting the release of the inodorous component 55 into the surrounding air.

Referring now to FIG. 6, another embodiment of a wetness indicating material 50b includes a pair of layers 63, 64 of a water-soluble substrate trapping the inodorous component 55 there between. The two substrate layers 63, 64 may be pre-formed with the inodorous component 55 there between, and the resulting assembly joined to other components of diaper 10. For example, and without limitation, the resulting assembly could then be joined to or otherwise disposed adjacent the optional acquisition material layer (not shown) forming part of the core 16, or the backsheet 12 or topsheet 14 of diaper 10. The location of wetness indicating material 50b is suitably chosen to be in or generally adjacent the core 16, so as to expose the material 50b to fluid secreted by the wearer. Exposure to urine, menses or other fluids secreted by the wearer is effective to dissolve or otherwise degrade the substrate layers 63, 64, thereby permitting the release of the inodorous component 55 into the surrounding air.

As stated above, system 4 (FIG. 1) includes a sensing device 6. Sensing device 6 is configured to detect the inodorous component 55 released into the surrounding air when the water-soluble element 60 has been dissolved or otherwise degraded by virtue of being exposed to urine, menses, or other fluids secreted by the wearer of diaper 10. In that regard, the sensing device 6 may be specifically configured to detect the presence of inodorous component 55 in the air, and to generate a signal in response to that detection. Suitable devices for sensing device 6 include, but are not limited to, combustion gas analyzers commercially available under model numbers 05500-00 and 05500-10, as well as combustible gas detectors commercially available under model number G-10350-00, and compact gas chromatographs sold under model numbers 34000-10 and 34000-30. All of the above exemplary devices are available from The Cole-Palmer Instrument Company, of Vernon Hills, Ill., United States of America. Similarly, a gas chromatograph commercially available under model number HP5990, from Agilent Technologies, Inc. of Santa Clara, Calif., United States of America, is also suitable for sensing device 6.

It is contemplated that sensing device 6 could be an apparatus configured to rest at a specified location, such as a tabletop or coupled to a bed rail, or may alternatively be a portable (e.g., wand-type) apparatus. For example, sensing device 6 could be portable so as to permit a caretaker to approach the wearer of diaper 10, and determine whether diaper 10 has been insulted without disturbing the wearer. Insofar as the released inodorous component 55 is undetectable by the human nose, the determination of whether diaper 10 has been insulted is also effected without the annoyance provided by conventional absorbent products releasing a particular human-detectable scent. A suitable portable apparatus for sensing device 6 is commercially available under model number GX-2012, available from RKI Instruments, Inc., of Union City, Calif., United States of America. Yet other suitable apparatus include detection of the inodorous component 55 by means of atomic absorption spectroscopy (AA), energy dispersive X-ray spectroscopy (EDS), or X-ray fluorescence spectroscopy (XRF).

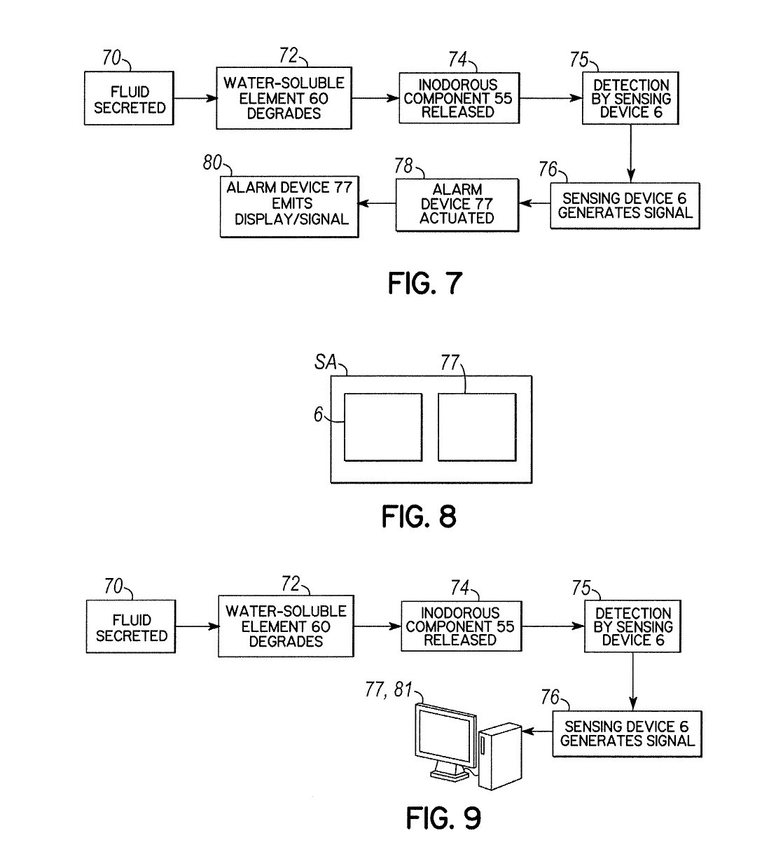

FIG. 7 illustrates an exemplary process for the detection of fluid secreted by the wearer of diaper 10. In that figure, block 70 corresponds to a fluid secretion event, such as urination or discharge of menses by the wearer of diaper 10. In block 72, the secreted fluid dissolves or otherwise degrades the water soluble element 60 of wetness indicating material 50. The dissolution/disintegration causes the inodorous component 55 to be released into the surrounding air (block 74). Sensing device 6 detects the presence of inodorous component 55 in the surrounding air and generates a signal (blocks 75, 76). The signal that is generated by sensing device 6 actuates an alarm device 77 (block 78), which emits, for example, a visual display, an audible signal, a vibratory signal, or any combination thereof (block 80). The signal or display emitted by the alarm device 77 lets the caretaker, or even the wearer himself/herself, know that the diaper 10 has been insulted with fluid secreted by the wearer.

It is contemplated that the sensing device 6 and the alarm device 77 may be integrated into a single apparatus SA, as schematically depicted in FIG. 8. Alternatively, the sensing device 6 and alarm device 77 may be apparatus that are separate from one another, and which may even be located remotely from one another. In that type of arrangement, schematically illustrated at FIG. 9, the alarm device 77 that is actuated by the signal generated by sensing device 6 (block 76) is located at a monitoring station 81, staffed by a caretaker or the like. This type of arrangement may be particularly useful in nursing homes, in which a staff member may be able to monitor, through monitoring station 81, any fluid-secreting occurrences among the many clients or patients living in the nursing home, all without the need to periodically physically inspect each client or patient for signs of wetness.

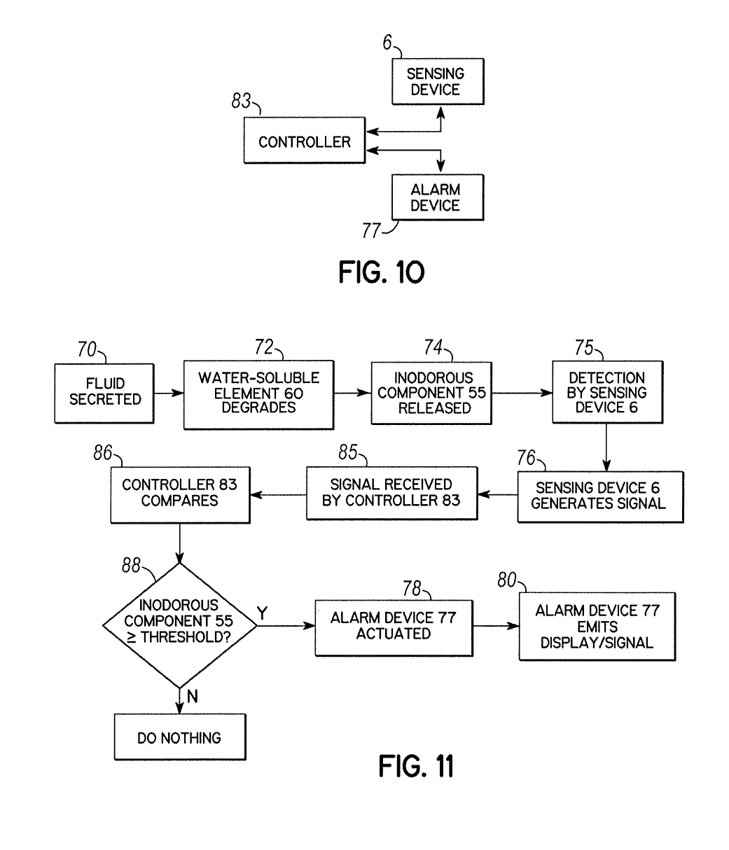

Referring now to FIGS. 10 and 11, a contemplated variation of system 4 includes a controller 83 that is operatively coupled to both, the sensing device 6, and the alarm device 77. For ease of understanding, like reference numerals in FIGS. 10 and 11 refer to similar features in the preceding figures, the description of which may be referred to for an understanding of the components and functionality of the embodiment of FIGS. 10 and 11 as well. In this embodiment, the controller 83 determines whether or not to generate a signal and thus actuate the alarm device 77 based on the signal received from the sensing device 6. More specifically, as illustrated in FIG. 11, the signal received by the controller 83 from sensing device 6 (block 85) is directly related to the amount of the inodorous component 55 detected by the sensing device 6. In that regard, the controller 83 compares (block 86) the amount of detected inodorous component 55 with a predetermined threshold. If the threshold is reached i.e., if the amount of detected inodorous component 55 is at least the threshold amount (block 88), the controller 83 generates a signal operative to actuate the alarm device 77 (block 78).

The exemplary detection process in FIG. 11 may be particularly useful for caretaking situations in which a small amount of fluid (e.g., urine, menses) secreted by the wearer is known not to be problematic. For example, there may be caretaking situations in which small amounts of fluid are well within the absorptive capacity of the diaper 10 (FIGS. 1-3), and are thereby not detrimental to the health of the wearer's skin. In that regard, the alarm device 77 will only be actuated, in such systems, when an amount of fluid has been secreted that necessitates replacement of the diaper 10 or, generally, the disposable absorbent product worn by the wearer.

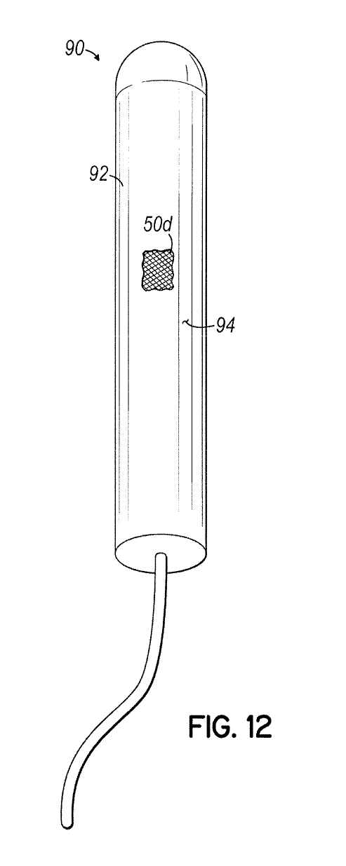

While the above description predominantly refers to the exemplary diaper 10 (FIGS. 1-3), it is understood that the present disclosure is similarly applicable to other types of disposable absorbent products. For example, tampons and other disposable catamenial or incontinence intravaginal devices are contemplated to fall within the scope of the present disclosure. FIG. 12 illustrates an exemplary tampon 90. Tampon 90 includes a wetness indicating material 50d (schematically depicted) similar to either of the wetness indicating material embodiments 50, 50a, 50b described with respect to the preceding figures. It is contemplated that the wetness indicating material 50d may be embedded in the interior of absorbent core 92 of the tampon 90, or be alternatively located on the surface 94 of the absorbent core 92. Further, the wetness indicating material 50d may be located in a component (not shown) of tampon 90, adjacent the absorbent core 92, so long as that component is exposed to fluid secreted by the wearer of the tampon 90.

Exemplary embodiments of the invention are described as follows, in non-limiting fashion:
1. A disposable absorbent product comprising:
   a topsheet;
   a backsheet overlaying said topsheet;
   an absorbent core disposed between said topsheet and said backsheet for absorbing fluid secreted by a wearer of the product; and
   a wetness indicating material in said topsheet, said backsheet, or said absorbent core, said wetness indicating material including a water-soluble element and an inodorous component trapped by said water-soluble element,
      wherein said water-soluble element is configured to degrade in the presence of fluid secreted by the wearer of the product, thereby releasing said inodorous component into the surrounding air.
2. The product of claim 1, wherein said wetness indicating material is embedded in said absorbent core.
3. The product of either of claim 1 or 2, wherein said water-soluble element includes a layer of a water-soluble substrate, said inodorous component being in or located adjacent said substrate.
4. The product of any of claims 1-3, wherein said water-soluble element includes first and second water-soluble substrates, said inodorous component being located between said first and second substrates.
5. The product of claim 1, wherein said inodorous component is encapsulated within shells of said water-soluble element.
6. The product of any of claims 1-5, wherein said inodorous component is a liquid at room temperature.
7. The product of any of claims 1-5, wherein said inodorous component is a gas at room temperature.
8. The product of claim 7, wherein said inodorous component is an inert gas.
9. The product of claim 7, wherein said inodorous component includes at least one gas selected from the group consisting of nitrogen, hydrogen, oxygen, methane, a nitrogen oxide, carbon dioxide, ethane, ethylene, propane, butane, isobutene, and acetylene.
10. The product of any of claims 1-9, wherein said water-soluble element includes a material selected from the group consisting of cyclodextrins, zeolites, and hemicarcerands.
11. A system for managing body waste, comprising:
   at least one disposable absorbent product that includes
   (a) a topsheet,
   (b) a backsheet overlaying said topsheet,
   (c) an absorbent core disposed between said topsheet and said backsheet for absorbing fluid secreted by a wearer of said at least one disposable absorbent product, and
   (d) a wetness indicating material in said topsheet, said backsheet, or said absorbent core, said wetness indicating material including a water-soluble element and an inodorous component trapped by said water-soluble element, said water-soluble element being configured to degrade in the presence of fluid secreted by the wearer, to thereby release said inodorous component into the surrounding air; and
   a sensing device in proximity with said disposable absorbent product, said sensing device being configured to detect the presence of said inodorous component in the surrounding air upon degradation of said water-soluble element and to generate a signal in response to said detection.
12. The system of claim 11, further comprising:
   an alarm device operatively coupled to said sensing device and actuatable in response to the signal generated by said sensing device.
13. The system of claim 12, wherein said sensing device and said alarm device form part of an integrated apparatus.
14. The system of claim 12, wherein said alarm device is located remotely from said sensing device.
15. The system of any of claims 12-14, wherein said alarm device is configured to emit, when actuated, at least one of a visual display, an auditory signal, or a vibratory signal.
16. The system of any of claims 12-15, further comprising:
   a controller operatively coupled to said sensing device and to said alarm device, said controller being configured to actuate said alarm device only when at least a predetermined threshold level of inodorous component has been detected by said sensing device.
17. The system of any of claims 11-16, wherein said inodorous component is a gas at room temperature, said gas being encapsulated within shells of said water-soluble element.
18. A disposable absorbent product comprising:
   a topsheet;
   a backsheet overlaying said topsheet;
   an absorbent core disposed between said topsheet and said backsheet for absorbing fluid secreted by a wearer of the product; and
   a wetness indicating material in said topsheet, said backsheet, or said absorbent core, said wetness indicating material including a water-soluble element and an inodorous gas encapsulated by said water-soluble element,
      wherein said water-soluble element is configured to dissolve in the presence of fluid secreted by the wearer of the product, thereby releasing said inodorous gas into the surrounding air.
19. The product of claim 18, wherein said gas is an inert gas.
20. The product of either of claim 18 or 19, wherein said water-soluble element includes a material selected from the group consisting of zeolites and hemicarcerands.
21. A disposable absorbent product comprising:
   an absorbent core configured to absorb fluid secreted by a wearer of the product; and
   a wetness indicating material in or adjacent said absorbent core, said wetness indicating material including a water-soluble element and an inodorous component trapped by said water-soluble element,
      wherein said water-soluble element is configured to degrade in the presence of fluid secreted by the wearer of the product, thereby releasing said inodorous component into the surrounding air.
      Yet other embodiments are also contemplated for uses and methods associated with any of the disposable absorbent products or systems according to any of claims 1 to 21, as described above. From the above disclosure of the general principles of the present invention and the preceding detailed description of exemplary embodiments, those skilled in the art will readily comprehend the various modifications to which this invention is susceptible. Accordingly, this invention is intended to be limited only by the scope of the following claims and equivalents thereof.

What is claimed is:

1. A disposable absorbent product comprising:
a topsheet;
a backsheet overlaying said topsheet;
an absorbent core disposed between said topsheet and said backsheet for absorbing fluid secreted by a wearer of the product; and
a wetness indicating material in said topsheet, said backsheet, or said absorbent core, said wetness indicating material including a water-soluble element and an inodorous component trapped by said water-soluble element,
wherein said water-soluble element is configured to degrade in the presence of fluid secreted by the wearer of the product, thereby releasing said inodorous component into the surrounding air.

2. The product of claim 1, wherein said wetness indicating material is embedded in said absorbent core.

3. The product of claim 1, wherein said water-soluble element includes a layer of a water-soluble substrate, said inodorous component being in or located adjacent said substrate.

4. The product of claim 1, wherein said water-soluble element includes first and second water-soluble substrates, said inodorous component being located between said first and second substrates.

5. The product of claim 1, wherein said inodorous component is encapsulated within shells of said water-soluble element.

6. The product of claim 1, wherein said inodorous component is a liquid at room temperature.

7. The product of claim 1, wherein said inodorous component is a gas at room temperature.

8. The product of claim 7, wherein said inodorous component is an inert gas.

9. The product of claim 7, wherein said inodorous component includes at least one gas selected from the group consisting of nitrogen, hydrogen, oxygen, methane, a nitrogen oxide, carbon dioxide, ethane, ethylene, propane, butane, isobutene, and acetylene.

10. The product of claim 1, wherein said water-soluble element includes a material selected from the group consisting of cyclodextrins, zeolites, and hemicarcerands.

11. A system for managing body waste, comprising:
at least one disposable absorbent product that includes
(a) a topsheet,
(b) a backsheet overlaying said topsheet,
(c) an absorbent core disposed between said topsheet and said backsheet for absorbing fluid secreted by a wearer of said at least one disposable absorbent product, and
(d) a wetness indicating material in said topsheet, said backsheet, or said absorbent core, said wetness indicating material including a water-soluble element and an inodorous component trapped by said water-soluble element, said water-soluble element being configured to degrade in the presence of fluid secreted by the wearer, to thereby release said inodorous component into the surrounding air; and
a sensing device in proximity with said disposable absorbent product, said sensing device being configured to detect the presence of said inodorous component in the surrounding air upon degradation of said water-soluble element and to generate a signal in response to said detection.

12. The system of claim 11, further comprising:
an alarm device operatively coupled to said sensing device and actuatable in response to the signal generated by said sensing device.

13. The system of claim 12, wherein said sensing device and said alarm device form part of an integrated apparatus.

14. The system of claim 12, wherein said alarm device is located remotely from said sensing device.

15. The system of claim 12, wherein said alarm device is configured to emit, when actuated, at least one of a visual display, an auditory signal, or a vibratory signal.

16. The system of claim 12, further comprising:
a controller operatively coupled to said sensing device and to said alarm device, said controller being configured to actuate said alarm device only when at least a predetermined threshold level of inodorous component has been detected by said sensing device.

17. The system of claim 11, wherein said inodorous component is a gas at room temperature, said gas being encapsulated within shells of said water-soluble element.

18. A disposable absorbent product comprising:
a topsheet;
a backsheet overlaying said topsheet;
an absorbent core disposed between said topsheet and said backsheet for absorbing fluid secreted by a wearer of the product; and
a wetness indicating material in said topsheet, said backsheet, or said absorbent core, said wetness indicating material including a water-soluble element and an inodorous gas encapsulated by said water-soluble element,
wherein said water-soluble element is configured to dissolve in the presence of fluid secreted by the wearer of the product, thereby releasing said inodorous gas into the surrounding air.

19. The product of claim 18, wherein said gas is an inert gas.

20. The product of claim 18, wherein said water-soluble element includes a material selected from the group consisting of zeolites and hemicarcerands.

21. A disposable absorbent product comprising:
an absorbent core configured to absorb fluid secreted by a wearer of the product; and
a wetness indicating material in or adjacent said absorbent core, said wetness indicating material including a water-soluble element and an inodorous component trapped by said water-soluble element,
wherein said water-soluble element is configured to degrade in the presence of fluid secreted by the wearer of the product, thereby releasing said inodorous component into the surrounding air.
